(19) United States
(12) Patent Application Publication
(10) Pub. No.: US 2022/0008252 A1
(43) Pub. Date: Jan. 13, 2022

(54) WIRELESS EXTRA-SENSORY LOCATION SUPPLEMENTARY FOR NAVIGATION OF THE VISUALLY IMPAIRED

(71) Applicant: Jacob Roman Donnini, Pilesgrove, NJ (US)

(72) Inventor: Jacob Roman Donnini, Pilesgrove, NJ (US)

(21) Appl. No.: 16/974,355

(22) Filed: Jul. 13, 2020

(51) Int. Cl. A61F 9/08; H04B 11/00; G06F 1/16; G01S 15/88; G01S 15/04

(52) U.S. Cl. CPC A61F 9/08; H04B 11/00; G01S 15/04; G01S 15/88; G06F 1/163

(57) ABSTRACT

A wearable device that pulses vibrators located on the user's back with different frequencies based on the distance of objects from the user. This distance is found via distance sensors on the hands and feet. The vibrating element that pulses is determined by the position of each sensor around the user's body. The combination of all these functions allow the wearer to get a sense of their surrounding without having to touch or see anything. The advantages of the device are a person with visual impairments can sense where things are without having to touch anything like all prior art requires and allow them to be totally immersed in their environment.

### FIELD

This disclosure generally relates to the field of computing devices. More particularly, the disclosure relates to wearable computing devices that assist visually impaired users.

### BACKGROUND

Visually impaired individuals, of whom there are upwards of 285 million worldwide, have long relied on objects like canes and guide dogs to assist with object detection and navigation. More modern systems often make use of more advanced technologies, such as an object detection device using ultrasonic transducers. Those devices may generate ultrasonic waves that produce an echo from a detected object. The echo is then detected and received by receivers that alert a visually impaired individual of objects in front of him or her. In those devices, because ultrasonic waves are undetectable by a human ear, a receiver, separate from the human ear, is needed to receive information about objects at a distance from a wearer.

Those sorts of devices often make use of the information encoded by the receiver to provide tactile or audio feedback to a user. For example, one “smart cane” employed provides a vibration to a user to indicate that a nearby object is detected in front of the cane user. Canes, however, whether “smart” or not typically have only one sensor and the user must still rely on the cane for physical contact of objects around the user, even if the objects may be delicate, dangerous or even other people. Additionally, carrying a large cane is cumbersome and a challenge to manage and is hard to carry during the day. Holding a cane requires a hand, limiting the user to only using one hand for other tasks. This invention aims to solve this issue in a high-tech innovative way.

### SUMMARY

Several objects and advantages of the present invention are:

To find the distance of objects from the user's limbs with a fair degree of accuracy.

To transmit the data gathered from all four sensors wirelessly to the main board located in the back of the user simultaneously.

To pulse vibrating elements with a frequency inversely proportional to the distance and can be modeled using this equation:

f=1/0.01D

Another object is to accomplish all of the above tasks but to change vibration location bases of the position of the sensors. For each sensor there will be three possible vibration locations that will be utilized based on the position of the sensor. For example, if the right-hand sensor is 45 degrees to the body then the middle sensor will vibrate with the distance feedback.

Yet another object is to accomplish the goals herein with the use of a garment such as, for example, a belt. The belt may have, for example, 12 vibrating elements and will strap around the waist with the main board on the back with the six vibrating elements on either side.

Still yet another object has the main board on the center of the back. There may be 12 total vibrating elements split into groups of three. Two can be located on either shoulder and the other two can be located just under each armpit.

Thus, in one aspect, disclosed herein is a wearable system for sensing objects for the visually impaired, the system comprising: a garment comprising a housing comprising a battery-powered receiver comprising electronic components, and an antenna, wherein the electronic components comprise a microcontroller on a circuit board, wherein the microcontroller comprises a wireless transceiver, and wherein the circuit board comprises a gyroscope, an accelerometer, and a magnetometer; at least one vibrating element on each side of the housing, wherein the at least one vibrating element is in communication with the microcontroller; an ultrasonic sensor unit for the right hand capable of transmitting signals to the receiver, wherein the ultrasonic sensor unit comprises a fastening device and a housing; an ultrasonic sensor unit for the left hand capable of transmitting signals to the receiver, wherein the ultrasonic sensor unit comprises a fastening device and a housing; an ultrasonic sensor unit for the right foot capable of transmitting signals to the receiver, wherein the ultrasonic sensor unit comprises a fastening device and a housing; an ultrasonic sensor unit for the left foot capable of transmitting signals to the receiver, wherein the ultrasonic sensor unit comprises a fastening device and a housing, wherein the housing of each ultrasonic sensor unit comprises a microcontroller, a transceiver, and at least one direction-sensor selected from the group consisting of a gyroscope, an accelerometer, and a magnetometer, wherein the transceiver in each of the ultrasonic sensor units is in wireless communication with the battery-powered receiver on the garment; the battery-powered receiver is capable of receiving signals from each of the ultrasonic sensor units via the antenna when the system is powered on, wherein each ultrasonic sensor can detect distance information from an object and send the distance information to the battery-powered receiver and then to the microcontroller, wherein the microcontroller sends a vibratory frequency signal to the at least one vibrating element to alert a wearer to the presence of the object.

In another aspect, disclosed herein is a wearable system for sensing objects for the visually impaired, the system comprising: an ultrasonic sensor unit for the right hand capable of transmitting signals to the receiver, wherein the ultrasonic sensor unit comprises a fastening device and a housing; an ultrasonic sensor unit for the left hand capable of transmitting signals to the receiver, wherein the ultrasonic sensor unit comprises a fastening device and a housing; an ultrasonic sensor unit for the right foot capable of transmitting signals to the receiver, wherein the ultrasonic sensor unit comprises a fastening device and a housing; an ultrasonic sensor unit for the left foot capable of transmitting signals to the receiver, wherein the ultrasonic sensor unit comprises a fastening device and a housing, wherein the housing of each ultrasonic sensor unit comprises at least one vibrating element, a microcontroller, and at least one direction-sensor selected from the group consisting of a gyroscope, an accelerometer, and a magnetometer, wherein each ultrasonic sensor can detect distance information of an object and each microcontroller can sends a vibratory frequency signal to the at least one vibrating element to alert a wearer to the presence of the object.

### BRIEF DESCRIPTION OF DRAWINGS

FIG. 1 is an illustration of an embodiment with the vibrators on a belt on a human figure with sensors on the hands-on feet;

FIG. 2 is an illustration of the embodiment in the form of a belt spread out front and back to show components;

FIG. 3 in an isometric illustration of the embodiment in the form of the belt while not on a human;

FIG. 4 is a block diagram of the components of the main back circuit board;

FIG. 5 is a top illustration of the angles of the arm that cause the location of the vibrations to change for that embodiment exemplified on the right hand;

FIG. 6 is a graph of the exponential function of the distance detected versus the frequency of the vibrational pulses;

FIG. 7 is an illustration of an embodiment with the vibrators on a vest located on the center of the upper back on a human figure with sensors on each limb;

FIG. 8 is a front and back illustration of the right-hand distance sensor modeled on a hand as it would be in use;

FIG. 9 is a front and back illustration of the left-hand distance sensor modeled on a hand as it would be in use;

FIG. 10 is a block diagram of the distance sensors of which there are four for each limb;

FIG. 11 is a top illustration of the left and right distance sensor attached to shoes;

FIG. 12 is a side illustration of a distance sensor mounted to a shoe;

FIG. 13 is a side illustration of a distance sensor that is designed to be mounted to a shoe;

FIG. 14 is a flow chart depicting the processes undergone by the code on the microcontroller of the receiver; and

FIG. 15 is a flow chart depicting the processes undergone by the code on the microcontroller of each distance sensor.

### DETAILED DESCRIPTION

Each embodiment referenced has examples in the drawings above. The invention will be described through the separate embodiments and it is to be understood that the invention is not limited to each separate embodiment. It instead comprises different combinations of embodiments or a single embodiment on its own. The invention will also cover all alternatives, modifications, and equivalents within the spirit and scope of the invention as defined by the claims. Furthermore, major concepts of the device will be explained thoroughly, while well-known methods such as: circuits, components and services will not be explained in the same amount of detail.

Disclosed herein is a wearable system for sensing objects for the visually impaired, the system comprising: a garment comprising a housing comprising a battery-powered receiver comprising electronic components, and an antenna, wherein the electronic components comprise a microcontroller on a circuit board, wherein the microcontroller comprises a wireless transceiver, and wherein the circuit board comprises a gyroscope, an accelerometer, and a magnetometer; at least one vibrating element on each side of the housing, wherein the at least one vibrating element is in communication with the microcontroller; an ultrasonic sensor unit for the right hand capable of transmitting signals to the receiver, wherein the ultrasonic sensor unit comprises a fastening device and a housing; an ultrasonic sensor unit for the left hand capable of transmitting signals to the receiver, wherein the ultrasonic sensor unit comprises a fastening device and a housing; an ultrasonic sensor unit for the right foot capable of transmitting signals to the receiver, wherein the ultrasonic sensor unit comprises a fastening device and a housing; an ultrasonic sensor unit for the left foot capable of transmitting signals to the receiver, wherein the ultrasonic sensor unit comprises a fastening device and a housing, wherein the housing of each ultrasonic sensor unit comprises a microcontroller, a transceiver, and at least one direction-sensor selected from the group consisting of a gyroscope, an accelerometer, and a magnetometer, wherein the transceiver in each of the ultrasonic sensor units is in wireless communication with the battery-powered receiver on the garment; the battery-powered receiver is capable of receiving signals from each of the ultrasonic sensor units via the antenna when the system is powered on, wherein each ultrasonic sensor can detect distance information from an object and send the distance information to the battery-powered receiver and then to the microcontroller, wherein the microcontroller sends a vibratory frequency signal to the at least one vibrating element to alert a wearer to the presence of the object.

In embodiments, the system disclosed herein comprises a garment. As such, the system disclosed herein is configured to be carried with a user (e.g., worn by the user, in proximity to the user) and, thus, at least one component of the system disclosed herein is at least in part integrated into a wearable garment, wherein the garment can comprise a top (e.g., shirt, vest, belt, strap, etc.), a bottom (e.g., pants, shorts, skirt, etc.), a backpack, an undergarment, and any other suitable form of garment. Additionally or alternatively, the at least one component of the system disclosed herein can be configured to be mechanically coupled to a wearable garment (e.g., retained in one or more pockets of the garment, attached by fasteners such as buttons, clips, magnets, and/or hook-and-loop fasteners, attached by adhesive, etc.). Additionally or alternatively, certain components of the system disclosed herein can be incorporated into one or more wearable devices (e.g., a limb-coupled wearable device such as a wristband or ankle band, etc.).

In preferred embodiments, the wearable garment at least partially encircles a torso or a waistline of the user.

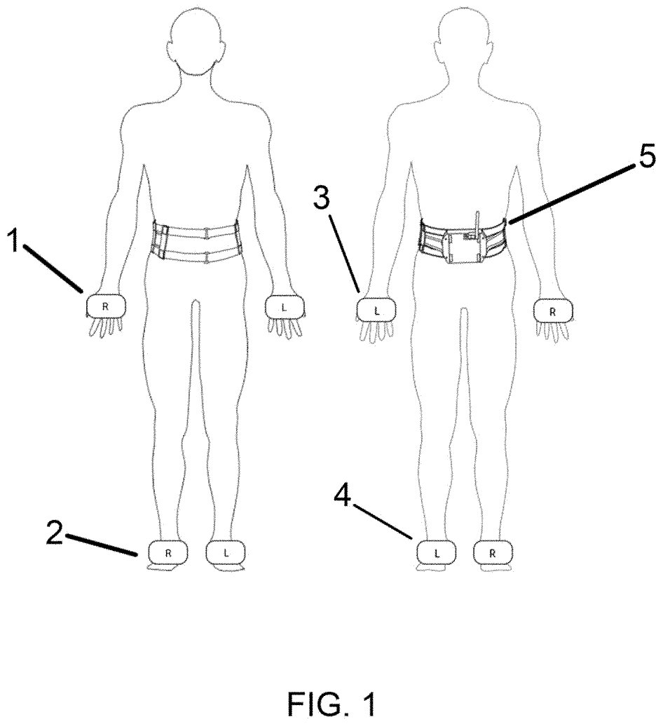

FIG. 1 depicts an embodiment of the present invention as it would be worn in use, wherein the garment is an adjustable belt or strap. Ultrasonic sensor units (also referred to herein as distance sensors or ultrasonic distance sensors) on the right hand 1, right foot 2, left hand 3 and left foot 4 send data to the receiver on the waist 5. In preferred embodiments, each ultrasonic distance sensor comprises an ultrasonic sensor, a gyroscope, an accelerometer, and a magnetometer. Shown here, the garment comprises a housing comprising a battery-powered receiver comprising electronic components, and an antenna, wherein the electronic components comprise a microcontroller on a circuit board, wherein the microcontroller comprises a wireless transceiver, and wherein the circuit board comprises a gyroscope, an accelerometer, and a magnetometer as will be described in more detail below.

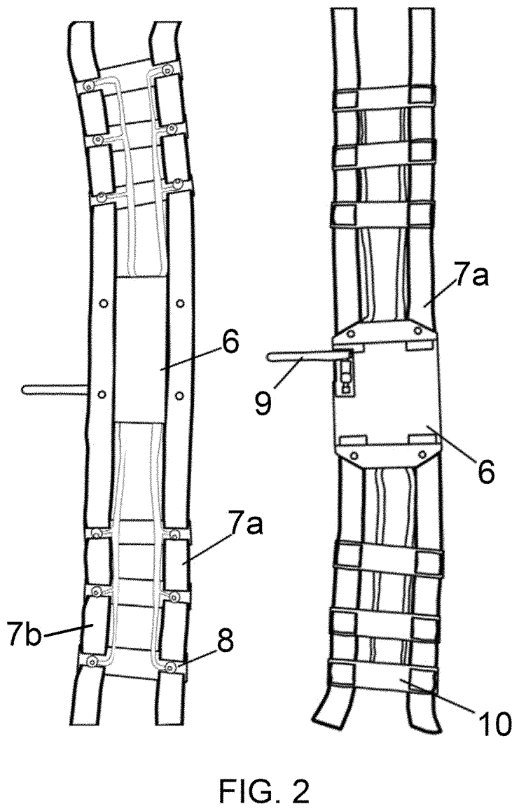

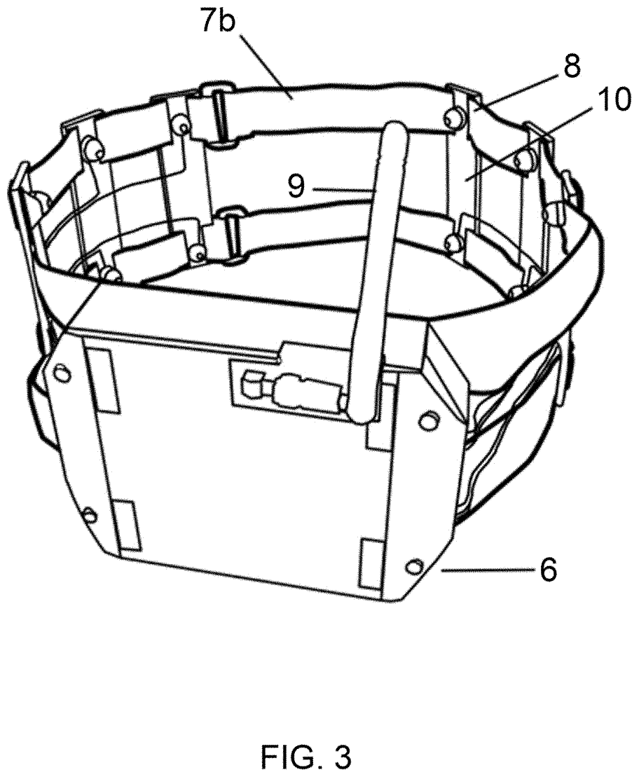

The main receiver, as shown in FIG. 2 and FIG. 3, comprises various electronic components, as well as straps and housing components, which can be 3D printed or injection molded pieces. The general design has a case that houses all the electronics in the center 6 with two Velcro straps 7a, 7b joined by three connectors 10 with vibrating elements 8 on them on either side of the case. The Velcro straps 7a, 7b are at least 100 cm long each and are generic Velcro with buckles on the end for fastening purposes.

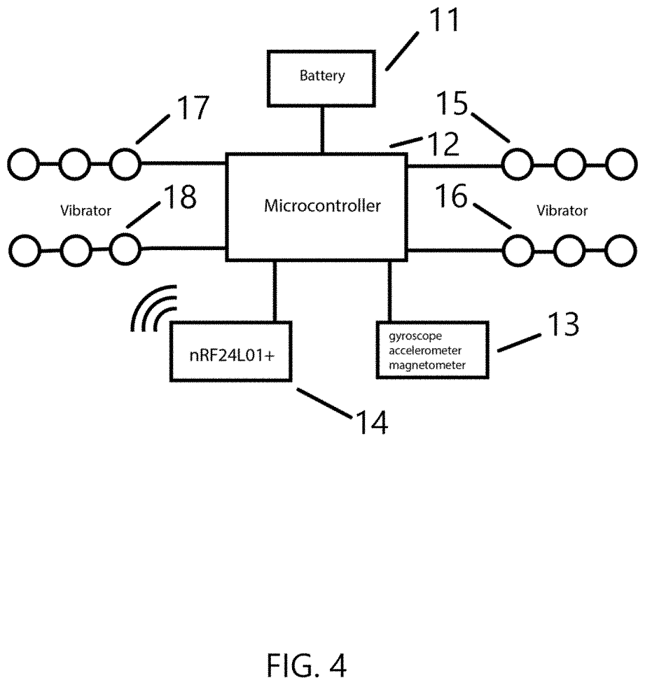

In this embodiment, the garment also comprises at least one vibrating element on each side of the housing, wherein the at least one vibrating element is in communication with the microcontroller; an ultrasonic sensor unit for the right hand capable of transmitting signals to the receiver, wherein the ultrasonic sensor unit comprises a fastening device; an ultrasonic sensor unit for the left hand capable of transmitting signals to the receiver, wherein each ultrasonic sensor unit comprises a fastening device. The vibrating elements 8 are standard shaftless vibration motors that are off-the-shelf components available at an online retailer. An antenna 9 protrudes from the center case and it is used to receive the wireless signals from the four sensors. The connectors 10 can be, for example, 3D printed objects using TPU so they are flexible and can conform to any body shape or they can be injection molded pieces. In FIG. 4 the electronics are powered by a 3.7v 1400 mah Li-ion rechargeable battery 11 and center around the main microcontroller 12 housed in a case 6 on a circuit board in the center of the device. In this embodiment, the microcontroller 12 is an Arduino Mega off-the-shelf variant that handles all the software and data processing of the main device. It also reads the MPU9250 board 13 located in the ultrasonic sensor units that hosts a gyroscope, accelerometer and magnetometer that are also on the same circuit board. The microcontroller also connects to the wireless transceiver 14 that reserves the incoming signals. The transceiver used in this embodiment is an NRF24L01+PA+LNA Wireless Transceiver which includes the antenna 9 and uses the 2.4 Gh band to communicate with the other sensors. In this embodiment, vibrating elements 8 connect directly to the microcontroller in four sets of three. The first set 15 are assigned to the right hand and the second set 16 are assigned to the right foot. On the left side, the first set 17 connect with the left hand and the second set 18 connect with the left foot. In this embodiment, the at least one vibrating element is/are positioned around the user's waist with the first nearest to the center on the back, second on the side and the third on the front to indicate direction.

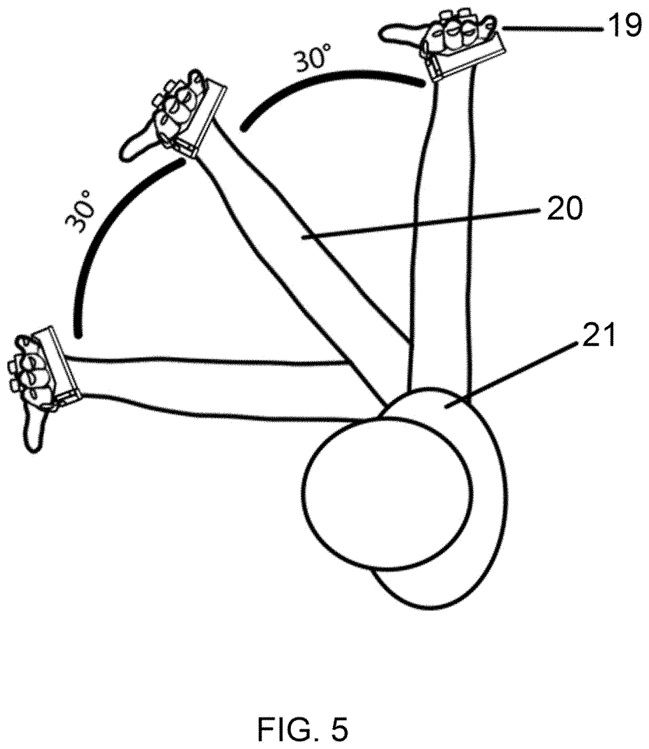

In this embodiment of the invention, the receiver is worn around the waist and the ultrasonic sensor units are worn on the limbs. The device can be used with one or more sensors connected at any time. Once powered on and connection is made, the user points the ultrasonic sensor unit unit in a direction and the vibrating elements on the receiver will vibrate at certain frequencies depending on the distance of objects in the surroundings. Depending on the direction in which the ultrasonic sensor unit is pointed, a different vibrating element or set of elements will activate. This is done to further immerse the user in their environment using the device. In FIG. 5 the drawing demonstrates three possible zones 30 degrees apart that the sensor 19 could be in relation to the body 21. These zones correspond to a set of three vibrating elements positioned on the receiver to give a better sense of where objects are in relation to the user. There could be at least one, two or up to three zones per sensor depending on the requirements of the device. Additionally, for more optional accuracy two additional gyroscopes can be placed on the elbow 20 in order to more accurately determine the sensor position using an inverse kinematic model or simple trigonometry. The receiver is also designed to work modularly with any one or more sensors at a time. This allows the user to customize their device and only use the sensors they need.

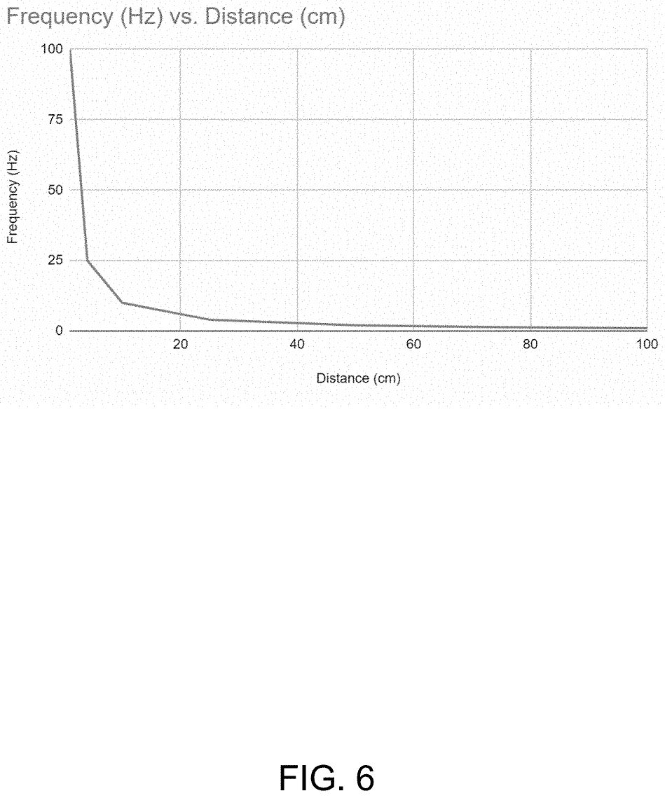

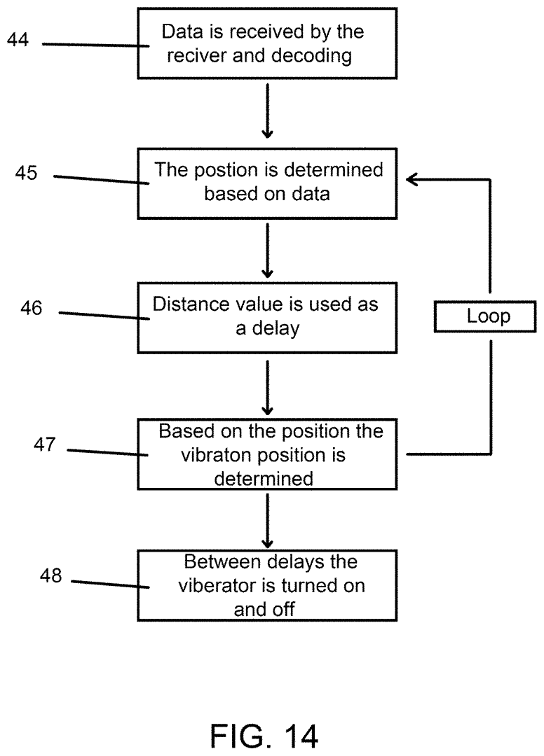

Regarding the software, each microcontroller's code is written in C++ and does a majority of the work of the embodiments disclosed herein. FIG. 14 depicts a flow chart of a visual representation of the code running on the microcontroller. The receiver's microcontroller reads local (i.e., same circuit board) sensor data, receives incoming data wirelessly from the ultrasonic sensor units, translates the distance data to vibrations and changes the location of the vibrations based on the location of the sensors. The code reads the data from the gyroscope(s), accelerometer, and magnetometer locally on the receiver and converts the raw data to degrees, meters per second squared and Tesla respectively. Optionally, it also reads the two additional gyroscopes on the elbows and does the same thing. Next, the receiver receives and decodes the wireless data from each ultrasonic sensor unit 44 simultaneously using an open source code library. This data pack consists of the gyroscope, accelerometer, and magnetometer data from each sensor and the distance found by the distance sensor. The code is able to change the position of the vibration to at least one other location based on the location of the sensors 45. Once this data is collected it then uses the distance found of an individual sensor 46 to find how long the delay between vibrations should be. It uses a timer that counts up and, by reducing the distance, it lowers the number that needs to be counted. At this point, based on the position of the distance sensor a vibration position is selected 47. These three steps are done respectively in a loop and turns a vibrating element or set of vibrating elements on and off with varying frequency 48. The end result is the smaller the distance, the greater the frequency. A graph of this trend is modeled in FIG. 6. This is done by using the accelerometer and gyroscope data from the sensors, if the sensor moves to the right or the left then moves to the next position or previous position. Additionally, if the magnetometer reading of the sensor is close to the magnetometer reading on the main board, then it returns to the first position. This can be used to prevent errors of the gyroscope from piling up over time. If the device is in the embodiment featuring the extra gyroscopes on the elbow, then for each arm three points in space are created. The first is at the main circuit board of the receiver, the second on the elbow, and the third from the sensor on the hand. Using these points, an inverse kinematic model of the arm is created and can be used to precisely point where the hands are. An inverse kinematic model is a well-known set of formulas used in many robotics projects that uses trigonometry to find the angle between points. The receiver also allows for any number of sensors to be connected at a time. It does this by reading where the wireless data came from and then sends that data to the preassigned vibration locations on the garment. That concludes the main functions of the code but it also includes data smoothing functions and number rounding that is less important to the understanding.

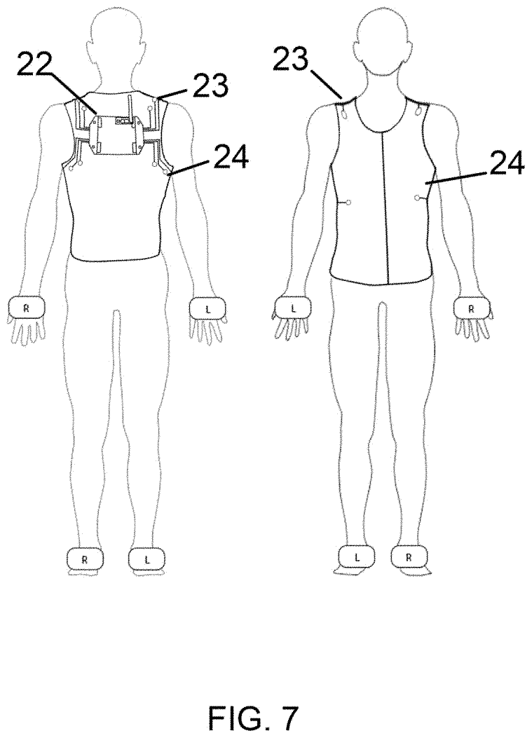

FIG. 7 shows an embodiment where the main circuit board of the receiver is positioned in the center of the back 22. In this configuration, the vibrating elements go over the shoulder 23 and under the armpit 24. This iteration takes on more of a vest formation rather than a belt. The advantages of this are that the shoulders and armpits are very sensitive, and the vibrations are felt more distinctly than on the belt.

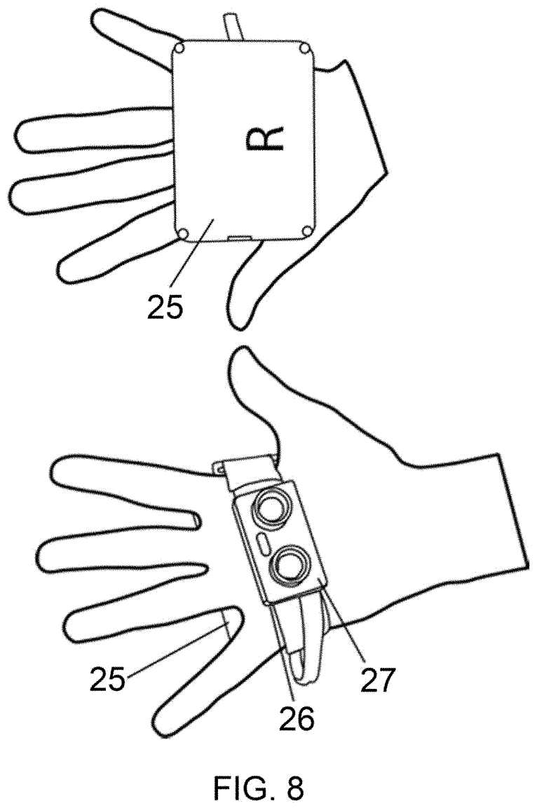

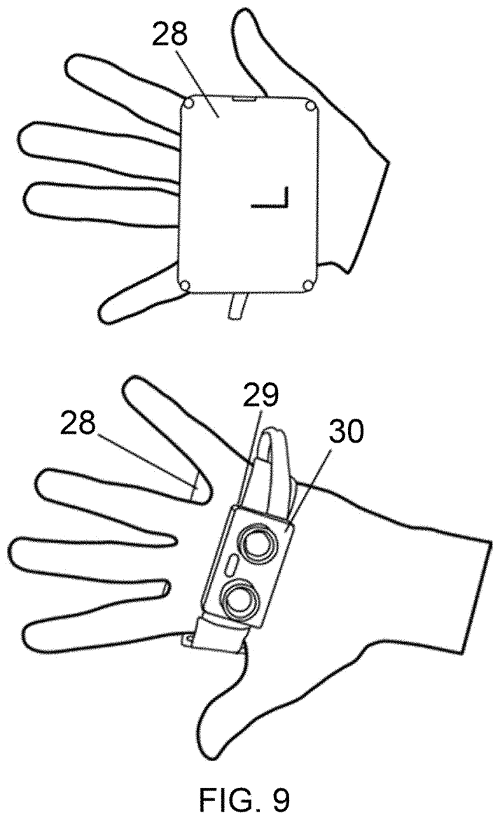

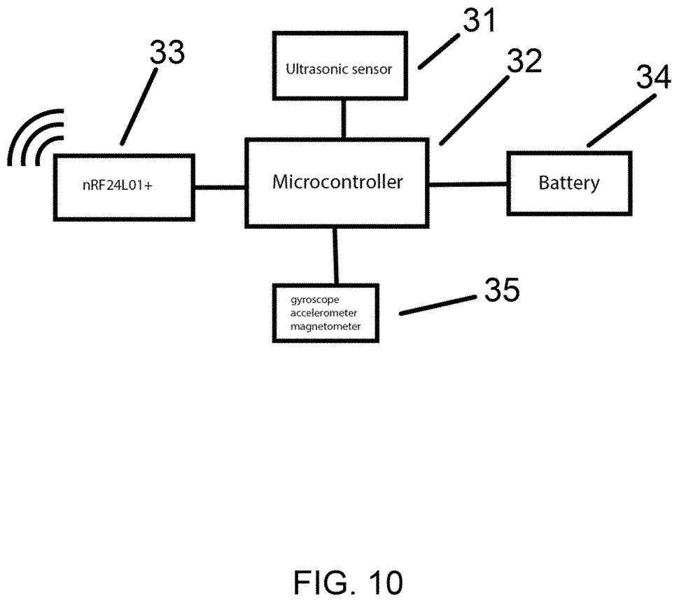

Referring now to FIG. 8 the ultrasonic sensor unit for the right hand is depicted. The ultrasonic sensor unit for the right hand is capable of transmitting signals to the receiver, wherein the ultrasonic sensor unit comprises a fastening device and a housing. The fastening device can be, for example, a Velcro strap. The housing of the ultrasonic sensor unit comprises a microcontroller, a transceiver, and at least one direction-sensor selected from the group consisting of a gyroscope, an accelerometer, and a magnetometer, wherein the transceiver in each of the ultrasonic sensor units is in wireless communication with the battery-powered receiver on the garment. The ultrasonic sensor unit comprises a housing 25 that holds the electronics worn on the back of the hand. On the palm of the hand, an ultrasonic sensor 26 and a sensor housing 27 that also holds the battery for the device. Referring to FIG. 9, the left-hand variant has the same hardware. The electronics housing 28, ultrasonic sensor 29 and sensor housing 30 are all mirrored to accommodate the left hand. Here, the ultrasonic sensor unit for the left hand is capable of transmitting signals to the receiver, wherein the ultrasonic sensor unit comprises a fastening device and a housing. The fastening device can be, for example, a Velcro strap. The housing of the ultrasonic sensor unit comprises a microcontroller, a transceiver, and at least one direction-sensor selected from the group consisting of a gyroscope, an accelerometer, and a magnetometer, wherein the transceiver in each of the ultrasonic sensor units is in wireless communication with the battery-powered receiver on the garment. The electronics for the sensors are shown in diagram form on FIG. 10. The distance sensors in all of the sensor units are ultrasonic sensors which work by sending out an ultrasonic blast and then listen for its echo and then using that to find the distance using this equation:

D=1/2TC

In the equation, D is the distance and T is the time elapsed between pulses. C is the speed of sound which is around 343 meters per second or 1125 feet per second depending on the temperature and humidity. The main distance sensor 31 is connected to the microcontroller 32 on the main circuit board of the device. The microcontroller is a generic Arduino Nano that could be found off the shelf. Also connected to the microcontroller is the Wireless Transceiver 33, specifically the nRF24L01+ Wireless Transceiver that connects wirelessly to the receiver. Powering the sensor is a 3.7v 600 mah Li-ion rechargeable battery 34 and charging protection circuit. The last component is the MPU9600 35 board that hosts a gyroscope, accelerometer and magnetometer used to find its position. This is the same board used on the receiver. Additionally, not featured in the diagram, is an extra vibrating unit that can be used in case there is no connection to the receiver.

The sensor is used by being worn on the hand with the distance sensor on the palm. The hand is then tilted up at a 90-degree angle with the sensor facing out and pointed to the front or side of the user to scan for objects in the way. If for some reason the transmitter or reservoir are disconnected or the user decides not to use the receiver, the hand sensor can activate a dormant vibrating element in the case on the hand to do the same job as the receiver. However, in this configuration there is only one possible position for the vibrations, that being on the back of the hand. In either configuration the sensor will tell the user through vibrational pulses the distance of objects perpendicular to the sensor.

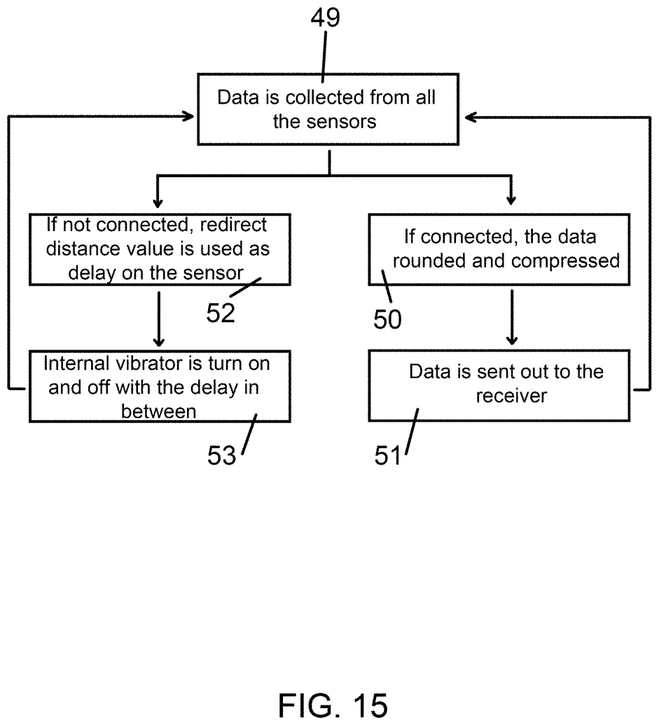

The software of the sensor units is responsible for transmitting the sensor data wirelessly, calculating the distance to the nearest whole centimeter and switching to an all-in-one mode in the event the signal is not being received. This is illustrated in FIG. 15 in a chart format. The sensor data is made up of the gyroscope, accelerometer, magnetometer and distance and is compressed and sent to the receiver 49. Once contact with the receiver is made it sends back a confirmation message to the transmitter and it sends the data repeatedly. This is all done using open source code libraries available in C++. Before the data is sent out, the ultrasonic distance sensor sends out an ultra-sonic blast and then starts a timer. When the blast bounces off an object and is received again the timer stops. That time is then multiplied by the speed of sound and divided in half. This results in the distance from the object, which is rounded to a whole number 50 and sent in the data structure along with the data from the other sensors 51. In the event that the data is not received, and the transmitter does not receive confirmation, the hand sensor will switch to an all-in-one mode 52. Thus, in another embodiment, the system disclosed herein does not include a receiver unit. This mode runs the same code loop from the receiver for the variable vibrational pulses but just uses the distance from the sensor and processes it locally instead of sending it to the receiver 53. Meanwhile it will continue to try to make contact with the receiver and once contact is re-established it will turn off the local vibrator and send data to the receiver again. That is the overview of the code that runs on the hand sensors.

Thus, in the alternate embodiments described in the preceding paragraph, disclosed is a wearable system for sensing objects for the visually impaired, the system comprising: an ultrasonic sensor unit for the right hand capable of transmitting signals to the receiver, wherein the ultrasonic sensor unit comprises a fastening device and a housing; an ultrasonic sensor unit for the left hand capable of transmitting signals to the receiver, wherein the ultrasonic sensor unit comprises a fastening device and a housing; an ultrasonic sensor unit for the right foot capable of transmitting signals to the receiver, wherein the ultrasonic sensor unit comprises a fastening device and a housing; an ultrasonic sensor unit for the left foot capable of transmitting signals to the receiver, wherein the ultrasonic sensor unit comprises a fastening device and a housing, wherein the housing of each ultrasonic sensor unit comprises at least one vibrating element, a microcontroller, and at least one direction-sensor selected from the group consisting of a gyroscope, an accelerometer, and a magnetometer, wherein each ultrasonic sensor can detect distance information of an object and each microcontroller can sends a vibratory frequency signal to the at least one vibrating element to alert a wearer to the presence of the object.

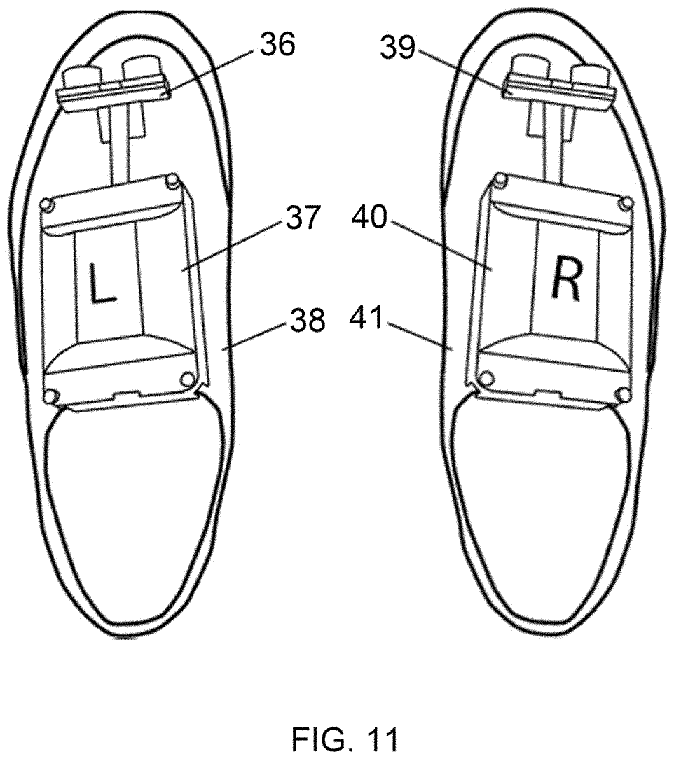

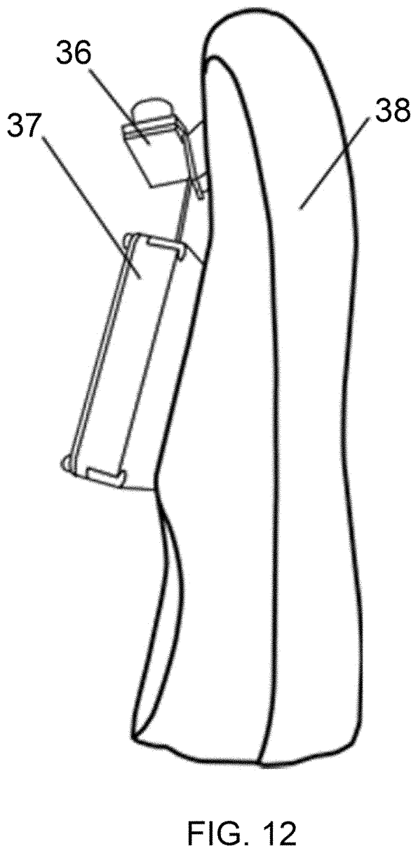

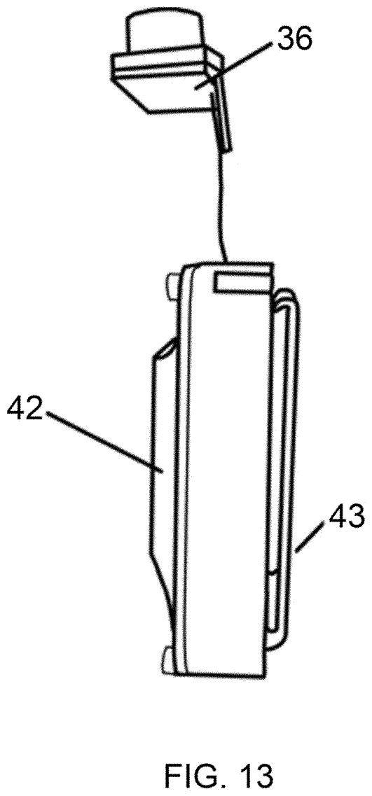

The next set of sensors are the distance sensors located on the feet, shown in FIG. 11. These include an ultrasonic sensor unit for the right foot capable of transmitting signals to the receiver, wherein the ultrasonic sensor unit comprises a fastening device and a housing; and an ultrasonic sensor unit for the left foot capable of transmitting signals to the receiver, wherein the ultrasonic sensor unit comprises a fastening device and a housing. The electronic hardware of the left foot sensor is identical to the hand sensors and are housed in a case 37 on top of the shoe 38. The differences are that the ultrasonic sensor is positioned at the toe 36 and fastened with Velcro, as well as minor changes to the electronics cases. Similarly, to the hand sensors, the right foot sensor's case 40 is the same as the left foot case except mirrored and on the right shoe 41. The ultrasonic sensor on the right foot 39 is also just a mirror image of its counterpart. In FIG. 12, the side view makes it clear where the electronics case attaches to the shoe. FIG. 13 shows the changes made to the electronics case from the hand sensor to the foot sensor. The first change is the bulge on the lid 42 that now houses the battery because there wasn't room on the ultrasonic sensor 36. The second change is the addition of the clip 43 that can be clipped into the shoelace. Because the clip is easy to attach and remove the foot sensor can be paired with any shoe with laces by simply adding a square of Velcro to the toe of the shoe and clipping the case to the laces.

The software for the foot sensors is identical to the hand sensors with a few noticeable exceptions. The first being the removal of the all-in-one feature that the hand sensors offer. This is not included in the foot sensors because it would not be easy to feel a vibration through a shoe, so they must be connected to the receiver to work. The second is there is no option for inverse kinematic positioning so the feet must rely on just the one gyroscope and magnetometer. Additionally, the left and the right foot sensors have unique vibration positions reserved on the receiver. On the belt, the right foot vibrating elements are directly below the right-hand vibrating elements and the left foot vibrating elements are directly below the left-hand vibrating elements. On the vest, the vibrators paired with the feet sensors go under the right and left armpit respectively. Other than those differences, the feet sensors function identically to the hand sensors.

I claim:

1. A wearable system for sensing objects for the visually impaired, the system comprising:
a. a garment comprising
i. a housing comprising a battery-powered receiver comprising electronic components, and an antenna, wherein the electronic components comprise a microcontroller on a circuit board, wherein the microcontroller comprises a wireless transceiver, and wherein the circuit board comprises a gyroscope, an accelerometer, and a magnetometer;
ii. at least one vibrating element on each side of the housing, wherein the at least one vibrating element is in communication with the microcontroller;
b. an ultrasonic sensor unit for the right hand capable of transmitting signals to the receiver, wherein the ultrasonic sensor unit comprises a fastening device and a housing;
c. an ultrasonic sensor unit for the left hand capable of transmitting signals to the receiver, wherein the ultrasonic sensor unit comprises a fastening device and a housing;
d. an ultrasonic sensor unit for the right foot capable of transmitting signals to the receiver, wherein the ultrasonic sensor unit comprises a fastening device and a housing;
e. an ultrasonic sensor unit for the left foot capable of transmitting signals to the receiver, wherein the ultrasonic sensor unit comprises a fastening device and a housing, wherein the housing of each ultrasonic sensor unit comprises a microcontroller, a transceiver, and at least one direction-sensor selected from the group consisting of a gyroscope, an accelerometer, and a magnetometer, wherein the transceiver in each of the ultrasonic sensor units is in wireless communication with the battery-powered receiver on the garment;
the battery-powered receiver is capable of receiving signals from each of the ultrasonic sensor units via the antenna when the system is powered on, wherein each ultrasonic sensor can detect distance information from an object and send the distance information to the battery-powered receiver and then to the microcontroller, wherein the microcontroller sends a vibratory frequency signal to the at least one vibrating element to alert a wearer to the presence of the object.

2. The wearable system of claim 1 wherein the garment is a belt.

3. The wearable system of claim 1 wherein the garment is a vest.

4. The wearable system of claim 1 wherein the garment is a shirt.

5. The wearable system of claim 1 wherein each fastening device is a Velcro strap.

6. The wearable system of claim 1 wherein the at least one vibrating element comprises a shaftless vibration motor.

7. The wearable system of claim 1 further comprising at least one additional sensor comprising a gyroscope.

8. The wearable system of claim 1 wherein each of the ultrasonic sensor unit for the right hand and the ultrasonic sensor unit for the left hand are locatable in a palm portion of a hand.

9. The wearable system of claim 1 wherein the at least one vibrating element comprises at least one vibrating element for each of the ultrasonic sensor units.

10. The wearable system of claim 9 wherein the at least one vibrating element comprises at least two vibrating elements for each of the ultrasonic sensor units.

11. The wearable system of claim 10 wherein the at least one vibrating element comprises at least three vibrating elements for each of the ultrasonic sensor units.

12. The wearable system of claim 1 wherein the microcontroller sends a vibratory higher frequency signal to the at least one vibrating unit to alert a wearer to the presence of the object as being at a closer distance to the wearer and sends a vibratory lower frequency signal to the at least one vibrating unit to alert a wearer to the presence of the object as being at a further distance to the wearer.

13. The wearable system of claim 1 wherein each ultrasonic sensor unit further comprises at least one vibrating element.

14. A wearable system for sensing objects for the visually impaired, the system comprising:
a. an ultrasonic sensor unit for the right hand capable of transmitting signals to the receiver, wherein the ultrasonic sensor unit comprises a fastening device and a housing;
b. an ultrasonic sensor unit for the left hand capable of transmitting signals to the receiver, wherein the ultrasonic sensor unit comprises a fastening device and a housing;
c. an ultrasonic sensor unit for the right foot capable of transmitting signals to the receiver, wherein the ultrasonic sensor unit comprises a fastening device and a housing;
d. an ultrasonic sensor unit for the left foot capable of transmitting signals to the receiver, wherein the ultrasonic sensor unit comprises a fastening device and a housing,
wherein the housing of each ultrasonic sensor unit comprises at least one vibrating element, a microcontroller, and at least one direction-sensor selected from the group consisting of a gyroscope, an accelerometer, and a magnetometer, wherein each ultrasonic sensor can detect distance information of an object and each microcontroller can sends a vibratory frequency signal to the at least one vibrating element to alert a wearer to the presence of the object.

15. The wearable system of claim 14 wherein each fastening device is a Velcro strap.

16. The wearable system of claim 14 wherein the at least one vibrating element comprises a shaftless vibration motor.

17. The wearable system of claim 14 further comprising at least one additional sensor comprising a gyroscope.

18. The wearable system of claim 14 wherein each of the ultrasonic sensor unit for the right hand and the ultrasonic sensor unit for the left hand are locatable in a palm portion of a hand.

19. The wearable system of claim 14 wherein the at least one vibrating element comprises at least one vibrating element for each of the ultrasonic sensor units.
